(19) United States
(12) Patent Application Publication
(10) Pub. No.: US 2008/0206324 A1
(43) Pub. Date: Aug. 28, 2008

(54) PELLETS HAVING AN ACTIVE COMPOUND MATRIX AND A POLYMER COATING, AND A PROCESS FOR THE PRODUCTION OF THE PELLETS

(75) Inventors: Andreas Gryczke, Riedstadt (DE); Hans-Ulrich Petereit, Darmstadt (DE); Christian Meier, Darmstadt (DE); Kathrin Nollenberger, Frankfurt (DE); Christian Brunnengraber, Lorsch (DE); Andreas Klosendorf, Bad Schwalbach (DE); Reinhard Menzel, Gross-Umstadt (DE)

Correspondence Address: OBLON, SPIVAK, MCCLELLAND MAIER & NEUSTADT, P.C., 1940 DUKE STREET, ALEXANDRIA, VA, 22314, US

(73) Assignee: Evonik Roehm GmbH, Darmstadt (DE)

(21) Appl. No.: 12/030,377

(22) Filed: Feb. 13, 2008

Related U.S. Application Data
(60) Provisional application No. 60/908,854, filed on Mar. 29, 2007.

(30) Foreign Application Priority Data
Feb. 22, 2007 (DE) 102007009243.3

(51) Int. Cl. A61K 9/48; A61K 9/14; A61K 9/32

(52) U.S. Cl. 424/463; 424/487; 424/482

(57) ABSTRACT

An active compound-containing pellet has a polymer coating of an anionic (meth)acrylate copolymer and a pharmaceutically active substance, embedded in a polymer matrix of one or more polymers, a particle size in the range from 300 to 1100 μm, a friability of at most 0.1%, measured using 200 g of pellets in a screening machine having a 200 μm screen, a screening diameter of 20 cm and 1.5 mm shaking amplitude at a shaking frequency of 50 l/sec for 10 min in the presence of six rubber cubes having a 1.8 cm edge length, with the proviso that the pellet releases no more than 10% of the active compound in a release test according to USP in artificial gastric juice at pH 1.2 after 120 min.

### BACKGROUND OF THE INVENTION

1. Field of the Invention

The present invention relates to pellets having an active compound matrix and a polymer coating, and a process for the production of the pellets.

2. Description of the Related Art

WO 01/68058 describes the use of a multilayer pharmaceutical form which is essentially constructed from a core containing a pharmaceutical active compound, an inner coating of a copolymer or a mixture of copolymers which comprises 85 to 98% by weight of free radical-polymerized C₁- to C₄-alkyl esters of acrylic or of methacrylic acid and 15 to 2% by weight of (meth)acrylate monomers having a quaternary ammonium group in the alkyl radical and an outer coating of a copolymer which comprises 75 to 95% by weight of free-radical polymerized C₁- to C₄-alkyl esters of acrylic or of methacrylic acid and 5 to 25% by weight of (meth)acrylate monomers having an anionic group in the alkyl radical.

The proportion of the outer coating should be in the range from 10 to 50% by weight based on the weight of the core containing the active compound and the inner coating.

US 2005/0191352 describes the production of extrudates containing pharmaceutical active compounds having controlled release of active compound by means of melt extrusion. In addition to the active compound, the mixtures to be extruded can contain polymers such as, for example, EUDRAGIT® RS, EUDRAGIT® NE or mixtures of these polymers. The extrusion preferably takes place in a twin-screw extruder. The extrudates discharged can be comminuted and shaped in the hot state by means of rotating knives to give cylindrical or alternatively to give spherical, ellipsoidal or lenticular particles. The active compound-containing particles thus obtained can be further processed, e.g. by filling into capsules, to give multiparticulate pharmaceutical forms.

EP 1 563 897 A1 describes a device for the production of rounded pellets (pelletizer). The device consists of an upstream feed arrangement for deformable material which is in particular fed from an extruder and a housing having a rotating cutting unit for cutting the material into material sections, and means for generating a stream of gas in the housing, by the action of which the material sections collide with a housing wall, undergoing rounding. Preferably, the housing wall is cooled in order to reduce the material removed. The device is suitable in particular for the production of pellets for the pharmaceutical sector by mixing pharmaceutical excipients, such as, for example, polymers, with at least one pharmaceutical active compound in the extruder, the extrudate emerging through a nozzle in the cutter housing and being comminuted and rounded by die-face cutting with gas cooling to give pellets.

Coated pharmaceutical forms are known, for example, from WO 01/68058. In WO 01/68058, application amounts generally from 10 to 50% by weight and in Examples 14 to 30% by weight, based on the weight of the core containing the active compound and the inner coating, are indicated for the outer polymer coating. The pharmaceutical form described in WO 01/68058 is complex in its production due to its layer structure.

### SUMMARY OF THE INVENTION

It is therefore an object of the present invention to provide coated pharmaceutical forms or their precursors which can be prepared more easily and cost-efficient.

This and other objects have been achieved by the present invention the first embodiment of which includes an active compound-containing pellet, comprising:

a polymer coating of an anionic (meth)acrylate copolymer; and

a pharmaceutically active substance, embedded in a polymer matrix of one or more polymers,

said pellet having
a particle size in the range from 300 to 1100 μm,
a friability of at most 0.1%, measured using 200 g of pellets in a screening machine having a 200 μm screen, a screening diameter of 20 cm and 1.5 mm shaking amplitude at a shaking frequency of 50 l/sec for 10 min in the presence of six rubber cubes having a 1.8 cm edge length,

with the proviso that the pellet releases no more than 10% of the active compound in a release test according to USP (United States Pharmacopeia) in artificial gastric juice at pH 1.2 after 120 min.

In another embodiment, the present invention provides a process for the production of an active compound-containing the above pellet by melt processing, comprising:

mixing the pharmaceutically active substance and the polymer(s) for the polymer matrix, to obtain a mixture; and

maintaining said mixture for at least 10 sec at a temperature of at least 5° C. above the glass transition temperature of the polymer or, in the case of a polymer mixture, based on the polymer having the highest glass transition temperature;

extruding said mixture in an extruder, to obtain an extruded mixture; and

discharging the extruded mixture by die-face cutting with subsequent rounding to give pellets having an average particle size in the range from 300 to 1100 μm; and

coating the pellet by spray application with a polymer coating of an anionic (meth)acrylate copolymer.

In another embodiment, the present invention relates to a method of delayed release of a pharmaceutically active substance, comprising:

administering a pellet as above to an organism in need thereof.

### DETAILED DESCRIPTION OF THE INVENTION

The object is achieved in particular by active compound-containing pellets having a polymer coating and an average particle size in the range from 300 to 1100 μm, comprising a pharmaceutically active substance, embedded in a polymer matrix of one or more polymers, characterized in that the pellets have a friability of at most 0.1%, measured using 200 g of pellets in a screening machine having a 200 μm screen, a screen diameter of 20 cm and 1.5 mm shaking amplitude at a shaking frequency of 50/sec for 10 min in the presence of six rubber cubes having a 1.8 cm edge length, and are coated with a polymer coating of an anionic (meth)acrylate copolymer, with the proviso that the pellets release no more than 10% of the active compound contained in the release test according to USP in artificial gastric juice at pH 1.2 after 120 min.

The invention furthermore relates to a process for the production of the pellets.

The invention furthermore relates to multiparticulate pharmaceutical forms, comprising one or more of the pellets according to the invention.

The invention relates to active compound-containing pellets having a polymer coating and an average particle size in the range from 300 to 1100 μm, comprising a pharmaceutically active substance, embedded in a polymer matrix of one or more polymers, characterized in that the pellets have a friability of at most 0.1%, measured using 200 g of pellets in a screening machine having a 200 μm screen, a screening diameter of 20 cm and 1.5 mm shaking amplitude at a shaking frequency of 50 l/sec (50 Hertz) for 10 min in the presence of six rubber cubes having a 1.8 cm edge length, and are coated with a polymer coating of an anionic (meth)acrylate copolymer, with the proviso that in the release test according to USP the pellets release no more than 10%, preferably no more than 7%, particularly preferably no more than 5%, in particular no more than 3% of the active compound contained in artificial gastric juice at pH 1.2 after 120 min.

Active Compound Release According to USP

The active compound release can be determined according to USP, in particular USP 28-NF23, General Chapter <711>, Dissolution, Apparatus 2 (Paddle), Method <724>“Delayed Release (Enteric Coated) Articles—General General Drug Release Standard”, Method B (100 rpm, 37° C.): The pellets are first tested for gastric juice resistance for 120 min in artificial gastric juice (USP) at pH 1.2. The active compound concentration in the test medium can be determined depending on the active compound, e.g. photometrically.

Average Particle Size

The average particle size of the pellets can be in the range from 300 to 1100, preferably from 400 to 1000 μm. The average particle size includes all values and subvalues therebetween, especially including 400, 500, 600, 700, 800, 900 and 1000 μm.

Active Compound Content

Based on a pellet without polymer coating, the active compound content can be 0.1 to 70, preferably 10 to 60, % by weight. The amount of active compound includes all values and subvalues therebetween, especially including 0.5, 1, 5, 10, 20, 30, 40, 50, and 60% by weight.

Friability

The pellets according to the invention have an extremely high abrasion resistance and friability. The abrasion resistance is markedly higher than in customary pharmaceutical forms or pellets and is barely even determinable in the standard test according to Ph. Eur. (5th Edition, Section 2.9.7) or less than 0.001%, that is, virtually zero. The standard test is therefore hardly suitable for the differentiation of the friability between pellets of the related art and the pellets according to the invention.

The friability of the pellets according to the invention is therefore described by a modified test having tightened conditions compared to the standard test.

The pellets have a friability of at most 0.1, preferably at most 0.05, %, measured using 200 g of pellets in a screening machine having a 200 μm screen, a screening diameter of 20 cm and 1.5 mm shaking amplitude at a shaking frequency of 50 l/s (50 Hertz) for 10 min in the presence of six rubber cubes having a 1.8 cm edge length (weight of the rubber cubes 8.3 g each). The friability includes all values and subvalues therebetween, especially including 0, 0.005, 0.01 and 0.05%.

The rubber cubes have an edge length of 1.8 cm and a weight of 8.3 g each; the density of the rubber is accordingly about 1.42 g/cm³. Rubber cubes of hard rubber are preferred.

The friability is determined in % by weighing the abraded material collected and setting it in proportion to the starting weight of the pellets.

For the measurement of the friability, it is possible, for example, to use a Retsch AS 200 Control screening machine. Screening machines of other manufacturers, using which the shaking conditions indicated can be produced, are equally suitable.

Polymer Matrix

The pellets according to the invention contain a pharmaceutically active substance, embedded in a polymer matrix of one or more polymers, preferably (meth)acrylate copolymers.

The proportion of the polymer matrix based on a pellet without polymer coating can be, for example, 20 to 99.9% by weight, preferably 30 to 60% by weight. The amount of polymer matrix includes all values and subvalues therebetween, especially including 20, 30, 40, 50, 60, 70, 80, 90, 95 and 99% by weight.

The polymer matrix can contain pharmaceutically customary excipients, e.g. binders.

(Meth)acrylate Copolymers in the Polymer Matrix

The polymer matrix can contain cationic (meth)acrylate copolymers, in particular (meth)acrylate copolymers having quaternary ammonium groups or (meth)acrylate-copolymers having tertiary ammonium groups.

The polymer matrix can contain completely or partially neutral or essentially neutral methacrylate copolymers or consist thereof.

(Meth)acrylate Copolymers Having Quaternary Ammonium Groups (EUDRAGIT® RS/RL Type)

Suitable polymers for the polymer matrix are, for example, (meth)acrylate copolymers having quaternary ammonium groups. (Meth)acrylate copolymers having quaternary ammonium groups are known, for example, from EP-A 181 515 or from DE-PS 1 617 751. Independently of the pH, these are water-insoluble or only water-swellable polymers, which are suitable for pharmaceutical coatings. A possible production process which may be mentioned is substance polymerization in the presence of a free radical-forming initiator dissolved in the monomer mixture. Likewise, the polymer can also be prepared by means of solution or precipitation polymerization. The polymer can in this way be obtained in the form of a fine powder, which is achievable in substance polymerization by grinding, in solution and precipitation polymerization, for example, by spray-drying.

The polymer matrix can contain a polymer of 98 to 85% by weight of C₁- to C₄-alkyl esters of acrylic or of methacrylic acid and 2 to 15% by weight of (meth)acrylate monomers having a quaternary ammonium group or a mixture of a number of polymers of this substance class.

Preferred C₁- to C₄-alkyl esters of acrylic or of methacrylic acid are methyl acrylate, ethyl acrylate, butyl acrylate, butyl methacrylate and methyl methacrylate.

As a (meth)acrylate monomer having quaternary ammonium groups, 2-trimethylammoniumethyl methacrylate chloride is particularly preferred.

The polymer matrix can contain a polymer of 93 to 88% by weight of C₁- to C₄-alkyl esters of acrylic or of methacrylic acid and 7 to 12% by weight of (meth)acrylate monomers having a quaternary ammonium group (EUDRAGIT® RL type).

A specifically suitable copolymer contains, for example, 60% by weight of methyl methacrylate, 30% by weight of ethyl acrylate and 10% by weight of 2-trimethylammoniumethyl methacrylate chloride (EUDRAGIT® RL).

The polymer matrix can contain a polymer of 97 to more than 93% by weight of C₁- to C₄-alkyl esters of acrylic or of methacrylic acid and 3 to less than 7% by weight of (meth)acrylate monomers having a quaternary ammonium group (EUDRAGIT® RS type).

A specifically suitable copolymer contains 65% by weight of methyl methacrylate, 30% by weight of ethyl acrylate and 5% by weight of 2-trimethylammoniumethyl methacrylate chloride (EUDRAGIT® RS).

In particular, mixtures of the (meth)acrylate copolymers of the EUDRAGIT® RL and EUDRAGIT® RS type mentioned are suitable, e.g. in the ratio from 20:1 to 1:20, preferably from 9:1 to 1:9, parts by weight. In particular, mixtures of EUDRAGIT® RSl and EUDRAGIT® RL are preferred.

Neutral (meth)acrylate Copolymers (EUDRAGIT® NE Type or EUDRAGIT® NM Type)

Further suitable polymers for the polymer matrix are, for example, neutral or essentially neutral methacrylate copolymers. Neutral or essentially neutral methacrylate copolymers can consist at least to 95, in particular to at least 98, preferably to at least 99, in particular to at least 99, particularly preferably to 100, % by weight of (meth)acrylate monomers having neutral radicals, in particular C₁- to C₄-alkyl radicals.

Suitable (meth)acrylate monomers having neutral radicals are, for example, methyl methacrylate, ethyl methacrylate, butyl methacrylate, methyl acrylate, ethyl acrylate, butyl acrylate. Methyl methacrylate, ethyl acrylate and methyl acrylate are preferred.

In small proportions, to less than 5, preferably at most 2, particularly preferably at most 1 or 0.05 to 1, % by weight, methacrylate monomers having anionic radicals, e.g. acrylic acid and/or methacrylic acid, can be contained.

Neutral or nearly neutral or essentially neutral (meth)acrylate copolymers of 20 to 40% by weight of ethyl acrylate, 60 to 80% by weight of methyl methacrylate and 0 to less than 5, preferably 0 to 2 or 0.05 to 1, % by weight (EUDRAGIT® NE type), for example, are suitable.

Nearly neutral or essentially neutral polymer exhibit a substantially pH independent behavior. The “essentially neutral” copolymers may contain small amounts of anionic monomers without changing the neutral character. Preferably, the “essentially neutral” copolymers may contain more than 95% by weight neutral monomer groups. In one embodiment, the “essentially neutral” copolymers may contain less than 5% anionic monomer groups. Anionic polymers may have 5 or more % by weight of ionic groups, preferably anionic groups.

EUDRAGIT® NE and EUDRAGIT® NM are copolymers of 30% by weight of ethyl acrylate and 70% by weight of methyl methacrylate.

Neutral or essentially neutral methyl acrylate copolymers which have been prepared according to WO 01/68767 as dispersions using 1-10% by weight of a non-ionic emulsifier having an HLB of 15.2 to 17.3 are preferred. The latter offer the advantage that phase separation with formation of crystal structures by the emulsifier is suppressed (EUDRAGIT® NM).

According to EP 1 571 164 A2, appropriate, nearly neutral (meth)acrylate copolymers, containing small proportions, 0.05 to 1% by weight, of monoolefinically unsaturated C3-C8-carboxylic acids, however, can also be prepared by emulsion polymerization in the presence of comparatively small amounts of anionic emulsifiers, e.g. 0.001 to 1% by weight.

(Meth)acrylate Copolymers Having Tertiary Amino Groups

Further suitable polymers for the polymer matrix are, for example, also (meth)acrylate copolymers having tertiary amino groups. (Meth)acrylate copolymers having tertiary amino groups can comprise partially or completely alkyl acrylates and/or alkyl methacrylates having a tertiary amino group in the alkyl radical. Suitable (meth)acrylate copolymers are known, for example, from EP 0 058 765 B1.

(Meth)acrylate copolymers having tertiary amino groups are only soluble in a pH range approximately below pH 5. They are therefore often used for the taste isolation of pharmaceutical forms or for pharmaceutical forms which are rapidly soluble in the gastric juice. In the context of the present invention, the polymer matrix, however, is coated with an anionic (meth)acrylate copolymer, which only dissolves in the intestinal juice, depending on type, from approximately 5.5 or thereover. In this pH range, (meth)acrylate copolymers having tertiary amino groups are insoluble or only swell. Like matrix polymers, they therefore have similarly release-delaying behaviour, like the (meth)acrylate copolymers having quaternary ammonium groups described above, and are thus a further alternative for the formulation of pellets and pharmaceutical forms according to the invention.

An appropriate (meth)acrylate copolymer can comprise, for example, 30 to 80% by weight of free radical-polymerized C₁- to C₄-alkyl esters of acrylic or of methacrylic acid and 70 to 20% by weight of (meth)acrylate monomers having a tertiary amino group in the alkyl radical (EUDRAGIT® E type).

Suitable monomers having functional tertiary amino groups are listed in U.S. Pat. No. 4,705,695, column 3, line 64 to column 4, line 13. Mention may be made in particular of dimethylaminoethyl acrylate, 2-dimethylaminopropyl acrylate, dimethylaminopropyl methacrylate, dimethylaminobenzyl acrylate, dimethylaminobenzyl methacrylate, (3-dimethylamino-2,2-dimethyl)propyl acrylate, dimethylamino-2,2-dimethyl)propyl methacrylate, (3-diethylamino-2,2-dimethyl)propyl acrylate and diethylamino-2,2-dimethyl)propyl methacrylate. Dimethylaminoethyl methacrylate is particularly preferred.

The content of the monomers having tertiary amino groups in the copolymers can advantageously be between 20 and 70% by weight, preferably between 40 and 60% by weight. The proportions of the C₁- to C₄-alkyl esters of the acrylic or methacrylic acid are 70-30% by weight. Mention may be made of methyl methacrylate, ethyl methacrylate, butyl methacrylate, methyl acrylate, ethyl acrylate and butyl acrylate.

A suitable (meth)acrylate copolymer having tertiary amino groups can be constructed, for example, of 20-30% by weight of methyl methacrylate, 20-30% by weight of butyl methacrylate and 60-40% by weight of dimethylaminoethyl methacrylate.

A specifically suitable commercially available (meth)acrylate copolymer having tertiary amino groups is constructed, for example, of 25% by weight of methyl methacrylate, 25% by weight of butyl methacrylate and 50% by weight of dimethylaminoethyl methacrylate (EUDRAGIT® E100 and EUDRAGIT® E PO (powder form)). EUDRAGIT® E100 and EUDRAGIT® E PO are water-soluble below about pH 5.0 and thus also gastric juice-soluble.

Polyvinyl Acetate/Polyvinyl Acetate Copolymers, Ethyl- and Methylcellulose

The polymer matrix can furthermore also contain a polyvinyl acetate, a polyvinyl acetate copolymer (e.g. KOLLICOAT® SR 30D or KOLLIDON® SR type), an ethylcellulose or a methylcellulose.

Polymer Coating

The active compound-containing pellets are coated with a polymer coating of an anionic (meth)acrylate copolymer.

The polymer coating can be, based on the pellet weight, 1 to 15, 1 to less than 14, preferably 1 to 13, particularly preferably 1 to less than 10, in particular 4 to 9, % by weight.

The polymer coating can contain pharmaceutically customary excipients, e.g. plasticizers.

The polymer coating can contain a polymer of 25 to 95% by weight of C₁- to C₄-alkyl esters of acrylic or of methacrylic acid and 5 to 75% by weight of (meth)acrylate monomers having an anionic group.

The polymer coating can contain a polymer of 40 to 60% by weight of methacrylic acid and 60 to 40% by weight of methyl methacrylate or 60 to 40% by weight of ethyl acrylate.

The polymer coating can contain a polymer of 20 to 40% by weight of methacrylic acid and 80 to 60% by weight of methyl methacrylate.

The polymer coating can contain a polymer of 10 to 30% by weight of methyl methacrylate, 50 to 70% by weight of methyl acrylate and 5 to 15% by weight of methacrylic acid.

(Meth)acrylate Copolymers Having Anionic Groups (EUDRAGIT® L, L100 55, S and FS Types)

Suitable anionic (meth)acrylate copolymers are polymers of 25 to 95% by weight of C₁- to C₄-alkyl esters of acrylic or of methacrylic acid and 5 to 75% by weight of (meth)acrylate monomers having an anionic group. Depending on the content of anionic groups and the character of the further monomers, appropriate polymers are water-soluble at pHs above pH 5.0 and thus also intestinal juice-soluble.

Usually, the proportions mentioned add up to 100% by weight. Additionally, however, without this leading to an adverse effect or change of the essential properties, small amounts in the range from 0 to 10, e.g. 1 to 5, % by weight of further vinylically copolymerizable monomers, such as, for example, hydroxyethyl methacrylate or hydroxyethyl acrylate, can be contained.

C₁- to C₄-alkyl esters of acrylic or methacrylic acid are in particular methyl methacrylate, ethyl methacrylate, butyl methacrylate, methyl acrylate, ethyl acrylate and butyl acrylate.

A (meth)acrylate monomer having an anionic group can be, for example, acrylic acid, but preferably methacrylic acid.

Furthermore, anionic (meth)acrylate copolymers of 40 to 60% by weight of methacrylic acid and 60 to 40% by weight of methyl methacrylate or 60 to 40% by weight of ethyl acrylate (EUDRAGIT® L or EUDRAGIT® L100-55 types) are suitable.

EUDRAGIT® L is a copolymer of 50% by weight of methyl methacrylate and 50% by weight of methacrylic acid.

EUDRAGIT® L100-55 is a copolymer of 50% by weight of ethyl acrylate and 50% by weight of methacrylic acid. EUDRAGIT® L 30D-55 is a dispersion comprising 30% by weight of EUDRAGIT® L 100-55.

Anionic (meth)acrylate copolymers of 20 to 40% by weight of methacrylic acid and 80 to 60% by weight of methyl methacrylate (EUDRAGIT® S type) are likewise suitable.

(Meth)acrylate copolymers consisting of 10 to 30% by weight of methyl methacrylate, 50 to 70% by weight of methyl acrylate and 5 to 15% by weight of methacrylic acid (EUDRAGIT® FS type) are particularly highly suitable.

EUDRAGIT® FS is a copolymer of 25% by weight of methyl methacrylate, 65% by weight of methyl acrylate and 10% by weight of methacrylic acid. EUDRAGIT® FS 30 D is a dispersion comprising 30% by weight of EUDRAGIT® FS.

Furthermore suitable for the purposes of the invention is a copolymer (see WO 2003/072087) which comprises
20 to 34% by weight of methacrylic acid and/or acrylic acid,
20 to 69% by weight of methyl acrylate and
0 to 40% by weight of ethyl acrylate and/or optionally
0 to 10% by weight of further vinylically copolymerizable monomers,
   with the proviso that the glass transition temperature of the copolymer according to ISO 11357-2, item 3.3.3, is at most 60° C. Because of its good elongation at break properties, this (meth)acrylate copolymer is suitable, in particular, for the compression of pellets to give tablets.

The copolymer in particular comprises free radical-polymerized units of

20 to 34, preferably 25 to 33, particularly preferably 28 to 32, % by weight of methacrylic acid or acrylic acid; methacrylic acid is preferred,

20 to 69, preferably 35 to 65, particularly preferably 35 to 55, % by weight of methyl acrylate and optionally

0 to 40, preferably 5 to 35, particularly preferably 15 to 35, % by weight of ethyl acrylate, with the proviso that the glass transition temperature of the copolymer (measurement without plasticizer addition at a residual monomer content (REMO) of less than 100 ppm, heating rate 10° C./min, nitrogen atmosphere) according to ISO 11357-2, item 3.3.3 (T_{mg}), is at most 60, preferably 40 to 60, particularly preferably 45 to 55° C.

The copolymer preferably consists essentially to exclusively of the monomers methacrylic acid, methyl acrylate and ethyl acrylate in the proportions indicated above.

Additionally, however, without this leading to an impairment of the essential properties, small amounts in the range from 0 to 10, e.g. 1 to 5, % by weight of further vinylically copolymerizable monomers, such as, for example, methyl methacrylate, butyl methacrylate, butyl acrylate or hydroxyethyl methacrylate, can be contained.

Glass transition temperature is understood here in particular as meaning the midpoint temperature T_{mg} according to ISO 11357-2, item 3.3.3. The measurement is carried out without plasticizer addition, at residual monomer contents (REMO) of less than 100 ppm, at a heating rate of 10° C./min and under a nitrogen atmosphere.

The copolymers are obtained in a manner known per se by free radical substance, solution, bead or emulsion polymerization. Before processing, they must be brought to the particle size range according to the invention by means of suitable grinding, drying or spraying processes.

This can be carried out by simple breaking of extruded and cooled granule strands or die-face cutting.

In particular on mixing with further powders or liquids, the use of powders can be advantageous. Suitable implements for the production of the powders are familiar to the person skilled in the art, e.g. air jet mills, pinned disc mills, fan mills. Optionally, appropriate screening steps can be included. A suitable mill for large industrial amounts is, for example, a counter jet mill (Multi No. 4200), which operates at about 6 bar overpressure.

Furthermore suitable for the purposes of the invention are copolymers (see WO 2004/096185) comprising
20 to 33% by weight of methacrylic acid and/or acrylic acid,
5 to 30% by weight of methyl acrylate and
20 to 40% by weight of ethyl acrylate and
greater than 10 to 30% by weight of butyl methacrylate and optionally
0 to 10% by weight of further vinylically copolymerizable monomers, where the proportions of the monomers add up to 100% by weight,
   with the proviso that the glass transition temperature of the copolymer according to ISO 11357-2, item 3.3.3 (midpoint temperature T_{mg}), is 55 to 70° C. Because of their good mechanical properties, copolymers of this type are in particular suitable for the compression of pellets to give tablets.

The abovementioned copolymer in particular comprises free radical-polymerized units of

20 to 33, preferably 25 to 32, particularly preferably 28 to 31, % by weight of methacrylic acid or acrylic acid; methacrylic acid is preferred,

5 to 30, preferably 10 to 28, particularly preferably 15 to 25, % by weight of methyl acrylate,

20 to 40, preferably 25 to 35, particularly preferably 28 to 32, % by weight of ethyl acrylate,

and greater than 10 to 30, preferably 15 to 25, particularly preferably 18 to 22, % by weight of butyl methacrylate,

where the monomer composition is chosen such that the glass transition temperature of the copolymer is 55 to 70° C., preferably 59 to 66, particularly preferably 60 to 65° C. Glass transition temperature is understood here in particular as meaning the midpoint temperature T_{mg} according to ISO 11357-2, item 3.3.3. Measurement is carried out without plasticizer addition, at residual monomer contents (REMO) of less than 100 ppm, at a heating rate of 10° C./min and under a nitrogen atmosphere.

The copolymer preferably consists essentially to exclusively, to 90, 95 or 99 to 100% by weight, of the monomers methacrylic acid, methyl acrylate, ethyl acrylate and butyl methacrylate in the proportions indicated above.

Additionally, however, without this having to lead to an impairment of the essential properties, small amounts in the range from 0 to 10, e.g. 1 to 5, % by weight of further vinylically copolymerizable monomers, such as, for example, methyl methacrylate, butyl acrylate, hydroxyethyl methacrylate, vinylpyrrolidone, vinylmalonic acid, styrene, vinyl alcohol, vinyl acetate and/or their derivatives, can be contained.

The copolymers are obtained in a manner known per se by free radical substance, solution, bead or emulsion polymerization. Before processing, they must be brought to the particle size range according to the invention by suitable grinding, drying or spraying processes.

This can be carried out by simple breaking of extruded and cooled granule strands or die-face cutting.

In particular on mixing with further powders or liquids, the use of powders can be advantageous. Suitable implements for the production of the powders are familiar to the person skilled in the art, e.g. air jet mills, pinned disc mills, fan mills. Optionally, appropriate screening steps can be included. A suitable mill for large industrial amounts is, for example, a counter jet mill (Multi No. 4200), which is operated at about 6 bar overpressure.

The production of the anionic (meth)acrylate copolymers containing proportions of anionic monomers of over 5% by weight in the polymer can be carried out in a manner known per se by free radical polymerization of the monomers (see, for example, EP 0 704 207 A2, EP 0 704 208 A2, WO 2003/072087, WO 2004/096185). The copolymers can be prepared in a manner known per se by free radical emulsion polymerization in aqueous phase in the presence of preferably anionic emulsifiers, for example according to the process described in DE-C 2 135 073.

The copolymers mentioned can be prepared continuously or batchwise (batch process) according to customary processes of free radical polymerization in the presence of free radical-forming initiators and optionally regulators for the adjustment of the molecular weight in substance, in solution, by bead polymerization or in emulsion. The average molecular weight Mw (weight average, determined, for example, by measurement of the solution viscosity) can be, for example, in the range from 80 000 to 1 000 000 (g/mol). Emulsion polymerization in aqueous phase in the presence of water-dissolved initiators and (preferably anionic) emulsifiers is preferred. In the case of substance polymerization, the copolymer can be obtained in solid form by breaking, extrusion, granulation or die-face cutting.

For the adjustment of special release profiles or sites of release, mixtures of the anionic (meth)acrylate copolymers mentioned can also be used. Optionally, mixtures of the anionic (meth)acrylate copolymers having no more than 50, preferably 10 to 30, % by weight of the already mentioned, neutral or essentially neutral methacrylate copolymers can also be present. Preferably the coatings, however, contain at most 10% by weight, preferably 0-5% by weight, in particular no, neutral or essentially neutral methacrylate copolymers.

Active Compounds

The pharmaceutically active substance obtained can be a pharmaceutical active compound or a food supplement.

One of the following pharmaceutically active substances can be contained: acamprosate, aceclofenac, acemetacin, acetylcysteine, acetylsalicylic acid, acetyltyrosine, acipimox, acitretin, alanine, alendronic acid, amethopterin, amino acids, amoxicillin, ampicillin, ascorbic acid, atorvastatin, azidocillin, aztreonam, bacampicillin, baclofen, benazepril, bendamustine, benzylpenicillin, bezafibrate, biotin, bornaprine, bumetanide, cabastine, canrenoic acid, carbamoylphenoxyacetic acid, carbidopa, carbimazole, carbocysteine, carisoprodol, cefaclor, cefadroxil, cefalexin, cefazoline, cefepime, cefetamet, cefixime, cefotaxime, cefotiam, cefoxitine, cefpodoxime, ceftazidime, ceftibutene, ceftriaxone, cefuroxime, cetirizine, chenodeoxycholic acid, chlorambucil, cidofovir, cilastatine, cilazapril, cinoxacin, ciprofloxacin, cisatracurium besilate, clavulanic acid, clodronic acid, clorazepate, cromoglicic acid, desmeninol, diclofenac, dicloxacillin, enoxacin, eprosartan, ethacrynic acid, etidronic acid, etofylline, etomidate, felbinac, felodipine, fenofibrate, fexofenadine, flavoxate, fleroxacin, flucloxacillin, flufenaminic acid, flumazenil, flupirtin, flurbiprofen, fluvastatin, fosfomycin, fosinopril, furosemide, fusidic acid, gabapentin, gemfibrozil, ibandronic acid, ibuprofen, iloprost, imidapril, imipenem, indomethacin, irinotecan, isradipine, ketoprofen, lercanidipine, levodopa, levofloxacin, liothyronine, lipoic acid, lisinopril, lodoxamide, lomefloxacin, lonazolac, loracarbef, loratadine, lovastatin, mefenamic acid, meropenem, mesalazine, metamizole, methotrexate, methyldopa, mezlocillin, moexipril, montelukast, moxifloxacin, mupirocin, naproxen, natamycin, nateglinide, nedocromil, nicotinic acid, nifedipine, nilvadipine, nimodipine, nisoldipine, nitrendipine, norfloxacin, ofloxacin, olsalazine, orotic acid, oxacillin, pamidronic acid, pangamic acid, penicillamine, phenoxymethylpenicillin, pentosan polysulphate, perindopril, pethidine, pipemidic acid, piperacillin, pirenoxine, piretanide, probenecid, proglumide, propicillin, prostaglandins, quinapril, quinaprilate, ramipril, repaglinide, reserpine, risedronic acid, salicylic acid, sulphasalazine, spirapril, sulbactam, sultamicillin, tazaroten, tazobactam, telmisartan, tiagabin, tiaprofenic acid, tilidine, tiludronic acid, trandolapril, tranexamic acid, valproic acid, vigabatrin, vincamine, vinpocetine, zanamivir, zoledronic acid, zopiclon, and their salts, isomers and combinations.

Process for the Production of the Pellets According to the Invention

The invention relates to a process for the production of active compound-containing pellets having a polymer coating by means of melt processing, where the pharmaceutically active substance and the polymer(s) for the polymer matrix are mixed and a temperature of at least 5° C. above the glass transition temperature of the polymer or, in the case of a polymer mixture, based on the polymer having the highest glass transition temperature, acts for at least 10 sec, preferably for at least 20 sec, the mixture is extruded in an extruder, preferably a twin-screw extruder, and discharged by die-face cutting with subsequent rounding to give pellets having a mean particle size in the range from 300 to 1100, preferably from 400 to 1000, μm, and the pellets are coated by means of spray application with a polymer coating of an anionic (meth)acrylate copolymer.

In the production of the pellets, pharmaceutically customary excipients can be added to the polymer matrix.

As a minimum requirement, a temperature of at least 5, preferably of at least 10, ° C. above the glass transition temperature of the polymer having the highest glass transition temperature should act on the mixture to be processed for at least 10, preferably for at least 20, sec. This causes the formation of a uniform melt phase.

Glass transition temperature is understood here in particular as meaning the midpoint temperature T_{mg} according to ISO 11357-2, item 3.3.3. The measurement is carried out without plasticizer addition, at residual monomer contents (REMO) of less than 100 ppm, at a heating rate of 10° C./min and under a nitrogen atmosphere. The glass transition temperature of EUDRAGIT® RS is approximately 50° C.

Under practical conditions, in many cases appropriate minimum temperatures are usually easily reached or exceeded and thus maintained over relatively long periods of time without this being critical for the pharmaceutically active substance or the polymers contained. Typical processing temperatures in the extruder can be, depending on the polymer composition of the mixture, for example, 50 to 200, preferably 100 to 180, ° C.

Depending on the mixture constituents, in particular the pharmaceutically active substances contained, care is to be taken, however, that temperatures and residence times are to be measured such that heat damage or adverse effects are avoided as far as possible. Usually, it will be attempted to set the processing temperatures and the residence times as low as possible first. With knowledge of the invention, a person skilled in the art can easily apply this to the individual case and proceed appropriately.

Application of the Polymer Coating

The polymer coatings on the active compound-containing pellets can be applied, for example, by spray application, preferably in fluidized bed apparatuses. The polymer coating is customarily mixed with plasticizers and release agents according to suitable processes. The polymer can be present here as a solution or suspension. The excipients can likewise be dissolved or suspended. Organic or aqueous solvents or dispersants can be used. For the stabilization of the dispersion, stabilizers can additionally be used (Example: polysorbate 80 or other suitable emulsifiers and stabilizers).

Examples of release agents are glycerol monostearate or other suitable fatty acid derivatives, silicic acid derivatives or talc. Examples of plasticizers are propylene glycol, phthalates, polyethylene glycols, sebacates or citrates, and other substances mentioned in the literature.

Multiparticulate Pharmaceutical Form

The pellets according to the invention can be contained in a multiparticulate pharmaceutical form, in particular in tablets, minitablets, capsules, sachets or inspissated juices.

A multiparticulate pharmaceutical form can contain as an individual dose, e.g. a capsule, expediently, for example, 20 to 1000 individual pellets. The pellets contained can be identical to one another and originate from a homogeneous pellet population. A number of pellet populations different from one another having different formulations can also be contained together in a multiparticulate pharmaceutical form.

The pellets according to the invention can thus be used for the production of pharmaceutical forms, in particular multiparticulate pharmaceutical forms.

Excipients

The polymer matrix and/or polymer coating contains pharmaceutically customary excipients.

In principle, of course, all excipients employed must be toxicologically harmless and suitable for the intended use, in particular in food supplements or pharmaceutical forms and without risk for the consumers or patients.

Amounts used and use of the customary additives in pharmaceutical coatings or coverings are familiar to the person skilled in the art. Customary additives can be, for example, release agents, pigments, stabilizers, antioxidants, pore formers, penetration promoters, lustering agents, flavourings or taste agents. They serve as processing aids and should guarantee a safe and reproducible production process and good long-term storage stability or they achieve additional advantageous properties in the pharmaceutical form. They are added to the polymer preparations before processing and can influence the permeability of the coatings, which can optionally be utilized as an additional control parameter.

Release Agents:

Release agents usually have lipophilic properties and are usually added to the spray suspensions. They prevent agglomeration of the cores during film-coating. Preferably, talc, Mg or Ca stearate, ground silicic acid, kaolin or nonionic emulsifiers having an HLB between 3 and 8 are employed. Customary amounts used for release agents are between 0.5 and 100% by weight based on the sum of active compound, water-soluble (meth)acrylate copolymer and water-insoluble polymer.

Pigments:

The pigments to be used are non-toxic and suitable for pharmaceutical purposes. For this see, for example, also: Deutsche Forschungsgemeinschaft, Farbstoffe fur Lebensmittel [German Research Association, colourants for foodstuffs], Harald Boldt Verlag KG, Boppard (1978); Deutsche Lebensmittelrundschau 74, No. 4, p. 156 (1978); Arzneimittelfarbstoffverordnung [Pharmaceutical Colourant Directive] AmFarbV of 25 Aug. 1980.

Suitable pigments are, for example, aluminium oxide pigments or Yellow Orange, Cochineal red lake, colour pigments based on aluminium oxide and azo dyes, sulphonic acid dyes, Yellow Orange S (E110, C.I. 15985, FD&C Yellow 6), Indigocarmine (E132, C.I. 73015, FD&C Blue 2), Tartrazine (E 102, C.I. 19140, FD&C Yellow 5), Ponceau 4R (E 125, C.I. 16255, FD&C Cochineal Red A), Quinoline Yellow (E 104, C.I. 47005, FD&C Yellow 10), Erythrosine (E127, C.I. 45430, FD&C Red 3), Azorubine (E 122, C.I. 14720, FD&C Carmoisine), Amaranth (E 123, C.I. 16185, FD&C Red 2), Brilliant Acid Green (E 142, C.I. 44090, FD&C Green S).

The E numbers of the pigments indicated refer to EU numbering. For this also see “Deutsche Forschungsgemeinschaft, Farbstoffe fur Lebensmittel”, Harald Boldt Verlag KG, Boppard (1978); Deutsche Lebensmittelrundschau 74, No. 4, p. 156 (1978); Arzneimittelfarbstoffverordnung AmFarbV of 25 Aug. 1980. The FD&C numbers refer to licensing in Food, Drugs and Cosmetics by the U.S. Food and Drug Administration (FDA) described in: U.S. Food and Drug Administration, Center for Food Safety and Applied Nutrition, Office of Cosmetics and Colors: Code of Federal Regulations—Title 21 Color Additive Regulations Part 82, Listing of Certified Provisionally Listed Colors and Specifications (CFR 21 Part 82).

Plasticizers

Further additives can also be plasticizers. Customary amounts are between 0 and 50, preferably 5 and 20, % by weight.

Depending on type (lipophilic or hydrophilic) and amount added, plasticizers can influence the functionality of the polymer layer. By physical interaction with the polymer, plasticizers achieve a lowering of the glass transition temperature and, depending on the amount added, promote film formation. Suitable substances usually have a molecular weight of between 100 and 20 000 and contain one or more hydrophilic groups in the molecule, e.g. hydroxyl, ester or amino groups.

Examples of suitable plasticizers are citric acid alkyl esters, glyceryl esters, phthalic acid alkyl esters, sebacic acid alkyl esters, sucrose esters, sorbitan esters, diethyl sebacate, dibutyl sebacate and polyethylene glycols 200 to 12 000. Preferred plasticizers are triethyl citrate (TEC), acetyltriethyl citrate (ATEC) and dibutyl sebacate (DBS). Mention may furthermore be made of esters which are usually liquid at room temperature, such as citrates, phthalates, sebacates or castor oil. Preferably, citric acid and sebacic acid esters are used.

The addition of the plasticizers to the formulation can be carried out in a known manner, directly, in aqueous solution or after heat pretreatment of a mixture. Mixtures of plasticizers can also be employed.

Coating of the Pellets

The film coatings on the active compound-containing pellets are customarily applied in fluidized bed apparatuses. The polymer coating is customarily mixed with plasticizers and release agents according to suitable processes. The polymer can be present as a solution or suspension here. The excipients can likewise be dissolved or suspended. Organic or aqueous solvents or dispersants can be used. For stabilization of the dispersion, stabilizers can additionally be used (Example: polysorbate 80 or other suitable emulsifiers and stabilizers).

Examples of release agents are glycerol monostearate or other suitable fatty acid derivatives, silicic acid derivatives or talc. Examples of plasticizers are propylene glycol, phthalates, polyethylene glycols, sebacates or citrates, and other substances mentioned in the literature.

Method of Delayed Release

The present invention also relates to a method of delayed release of a pharmaceutically active substance (or compound), comprising administering a pellet according to invention to an organism in need thereof. An organism in need of the pharmaceutically active compound includes humans and mammals.

Having generally described this invention, a further understanding can be obtained by reference to certain specific examples which are provided herein for purposes of illustration only, and are not intended to be limiting unless otherwise specified.

### EXAMPLES

All examples were extruded on an asynchronous 18 mm twin-screw extruder having a functional length of the process part of 40D. The extrusion temperature in the entry area was 10° C.-100° C.; in the following cylinders of the extruder the temperature was increased to 160° C. The melt was discharged at 160° C. and cut in an air-cooled die-face cutting process to give pellets. For the die-face cutting process, the melt was fed at the end of the extrusion process part into a conical melt channel, which at the end had a number of outlet openings at the base in the form of a ring. Above this ring rotate one or more knives, which cut off the melt in the hot state. The pellets were cooled in a stream of air and transported away. The rounding of the pellets was carried out by means of the surface tension still present in the melt and not or only to a very small extent during the transport of the pellets directly after the cutting process. The active compound and the polymers were fed to the extruder by means of gravimetric metering.

The film-coating of the pellets was carried out in a fluidized bed apparatus equipped as a bottom spray. The batch size was 100 g pellets.

### Example C1 is a Comparative Example

The pellets of Example C1 contained the water-insoluble polymer EUDRAGIT® RL and EUDRAGIT® RS. The pellets were film-coated with 1% by weight, based on polymer dry matter, of a suspension comprising the gastric juice-resistant polymer EUDRAGIT® L 30 D-55. The formulation showed no gastric juice resistance, since after 120 min at pH 1.2 16.8% of the initially contained active compound was already released.

The spray suspension for the film-coating was prepared as a 30% strength suspension, comprising EUDRAGIT® L 30 D-55, 10% of triethyl citrate based on 100% of polymer solid, 3% of glycerol monostearate based on 100% of polymer solid and 40% of polysorbate 80 based on 100% of glycerol monostearate.

### Examples 2 to 5 are Examples According to the Invention

The pellets of Examples 2 to 5 contained the water-insoluble polymer EUDRAGIT® RL and EUDRAGIT® RS. The pellets were film-coated with 2% to 6% (% by weight) of a suspension comprising the gastric juice-resistant polymer EUDRAGIT® L 30 D-55. The formulations showed gastric juice resistance, since after 120 min at pH 1.2 less than 10% of the initially contained active compound was released.

The spray suspension for the film-coating was prepared as a 30% strength suspension, comprising EUDRAGIT® L 30 D-55, 10% of triethyl citrate based on 100% of polymer solid, 3% glycerol monostearate based on 100% of polymer solid and 40% of polysorbate 80 based on 100% of glycerol monostearate.

| | Example No. | | | | |
|---|---|---|---|---|---|
| | C1 | 2 | 3 | 4 | 5 |
| Theophylline [% by weight] | 30 | 30 | 30 | 30 | 30 |
| EUDRAGIT ® RL [% by | 35 | 35 | 35 | 35 | 35 |
| weight] | | | | | |
| EUDRAGIT ® RS [% by | 35 | 35 | 35 | 35 | 35 |
| weight] | | | | | |
| EUDRAGIT ® L 30 D-55 film | 1 | 2 | 3 | 4 | 6 |
| application [% by weight] | | | | | |

| pH of the | | | | | | |
|---|---|---|---|---|---|---|
| medium | Time [min] | Active compound release [%] | | | | |
| 1.2 | 0 | 0.0 | 0.0 | 0.0 | 0.0 | 0.0 |
| 1.2 | 15 | 2.2 | 0.6 | 0.2 | 0.0 | 0.0 |
| 1.2 | 30 | 4.2 | 1.2 | 0.5 | 0.2 | 0.1 |
| 1.2 | 60 | 8.2 | 2.6 | 1.2 | 0.4 | 0.3 |
| 1.2 | 90 | 12.6 | 4.3 | 1.9 | 0.7 | 0.4 |
| 1.2 | 120 | 16.8 | 6.0 | 2.7 | 0.9 | 0.6 |
| 6.8 | 130 | 20.8 | 14.1 | 11.6 | 1.4 | 0.9 |
| 6.8 | 140 | 24.1 | 19.2 | 17.0 | 2.4 | 1.2 |
| 6.8 | 165 | 30.6 | 27.8 | 25.7 | 18.0 | 15.8 |
| 6.8 | 180 | 33.9 | 31.8 | 29.7 | 21.7 | 19.6 |
| 6.8 | 210 | 39.7 | 38.6 | 36.4 | 26.3 | 24.3 |
| 6.8 | 240 | 44.8 | 44.2 | 41.9 | 30.3 | 28.2 |
| 6.8 | 270 | 49.3 | 49.1 | 46.8 | 37.0 | 34.9 |
| 6.8 | 300 | 53.4 | 53.5 | 51.1 | 42.5 | 40.4 |
| 6.8 | 330 | 57.3 | 57.4 | 55.0 | 47.4 | 45.2 |
| 6.8 | 360 | 60.7 | 61.0 | 58.5 | 51.7 | 49.5 |
| 6.8 | 420 | 67.2 | 67.5 | 64.9 | 59.3 | 57.0 |
| 6.8 | 480 | 72.7 | 73.0 | 70.4 | 65.7 | 63.4 |

German patent application 10 2007 009 243.3 filed Feb. 22, 2007, and U.S. provisional application Ser. No. 60/908,854, filed Mar. 29, 2007, are incorporated herein by reference.

Numerous modifications and variations on the present invention are possible in light of the above teachings. It is therefore to be understood that within the scope of the appended claims, the invention may be practiced otherwise than as specifically described herein.

1. An active compound-containing pellet, comprising:
a polymer coating of an anionic (meth)acrylate copolymer; and
a pharmaceutically active substance, embedded in a polymer matrix of one or more polymers,
said pellet having
a particle size in the range from 300 to 1100 μm,
a friability of at most 0.1%, measured using 200 g of pellets in a screening machine having a 200 μm screen, a screening diameter of 20 cm and 1.5 mm shaking amplitude at a shaking frequency of 50 l/sec for 10 min in the presence of six rubber cubes having a 1.8 cm edge length,
with the proviso that the pellet releases no more than 10% of the active compound in a release test according to USP in artificial gastric juice at pH 1.2 after 120 min.

2. The active compound-containing pellet according to claim 1, wherein an amount of polymer coating is 1 to 15% by weight based on the pellet weight.

3. The active compound-containing pellet according to claim 1, wherein the active compound proportion based on a pellet without a polymer coating is 0.1 to 70% by weight.

4. The active compound-containing pellet according to claim 1, wherein the proportion of the polymer matrix based on a pellet without a polymer coating is 20 to 99.9% by weight.

5. The active compound-containing pellet according to claim 1, wherein the polymer matrix and/or polymer coating comprises at least one pharmaceutically customary excipient.

6. The active compound-containing pellet according to claim 1, wherein the polymer matrix comprises
a polymer comprising 98 to 85% by weight of a C₁- to C₄-alkyl ester of acrylic or of methacrylic acid; and
2 to 15% by weight of a (meth)acrylate monomer having a quaternary ammonium group or a mixture of at least two (meth)acrylate monomers each having a quaternary ammonium group.

7. The active compound-containing pellet according to claim 1, wherein the polymer matrix comprises a polymer comprising
93 to 88% by weight of a C₁- to C₄-alkyl ester of acrylic or of methacrylic acid; and
7 to 12% by weight of a (meth)acrylate monomer having a quaternary ammonium group.

8. The active compound-containing pellet according to claim 1, wherein the polymer matrix comprises a polymer comprising
97 to more than 93% by weight of a C₁- to C₄-alkyl ester of acrylic or of methacrylic acid; and
3 to less than 7% by weight of a (meth)acrylate monomer having a quaternary ammonium group.

9. The active compound-containing pellet according to claim 1, wherein a mixture of at least one polymer a) and at least one polymer b);
wherein a ratio of polymer a) to polymer b) is 20:1 to 1:20;
wherein polymer a) comprises
98 to 85% by weight of a C₁- to C₄-alkyl ester of acrylic or of methacrylic acid, and
2 to 15% by weight of a (meth)acrylate monomer having a quaternary ammonium group or a mixture of at least two (meth)acrylate monomers each having a quaternary ammonium group; and
wherein polymer b) comprises
97 to more than 93% by weight of a C₁- to C₄-alkyl ester of acrylic or of methacrylic acid; and
3 to less than 7% by weight of a (meth)acrylate monomer having a quaternary ammonium group.

10. The active compound-containing pellet according to claim 1, wherein the polymer matrix comprises a copolymer comprising
20 to 40% by weight of ethyl acrylate; and
60 to 80% by weight of methyl methacrylate; and
0 to less than 5% by weight of acrylic acid and/or methacrylic acid.

11. The active compound-containing pellet according to claim 1, wherein the polymer matrix comprises a polymer comprising
30 to 80% by weight of a C₁- to C₄-alkyl ester of acrylic or of methacrylic acid; and
70 to 20% by weight of a (meth)acrylate monomer having a tertiary amino group in the alkyl radical.

12. The active compound-containing pellet according to claim 1, wherein the polymer matrix comprises a member selected from the group consisting of a polyvinyl acetate, a polyvinyl acetate copolymer, an ethylcellulose, a methylcellulose and mixtures thereof.

13. The active compound-containing pellet according to claim 1, wherein the polymer coating comprises a polymer comprising
25 to 95% by weight of a C₁- to C₄-alkyl ester of acrylic or of methacrylic acid; and
5 to 75% by weight of a (meth)acrylate monomer having an anionic group.

14. The active compound-containing pellet according to claim 1, wherein the polymer coating comprises a polymer comprising
40 to 60% by weight of methacrylic acid; and
60 to 40% by weight of methyl methacrylate or 60 to 40% by weight of ethyl acrylate.

15. The active compound-containing pellet according to claim 1, wherein the polymer coating comprises a polymer comprising
20 to 40% by weight of methacrylic acid; and
80 to 60% by weight of methyl methacrylate.

16. The active compound-containing pellet according to claim 1, wherein the polymer coating comprises a polymer comprising
10 to 30% by weight of methyl methacrylate,
50 to 70% by weight of methyl acrylate, and
5 to 15% by weight of methacrylic acid.

17. The active compound-containing pellet according to claim 1, wherein the pharmaceutically active substance is a pharmaceutical active compound or a food supplement.

18. The active compound-containing pellet according to claim 1, wherein comprising a pharmaceutically active substance selected from the group consisting of acamprosate, aceclofenac, acemetacin, acetylcysteine, acetylsalicylic acid, acetyltyrosine, acipimox, acitretin, alanine, alendronic acid, amethopterin, amino acids, amoxicillin, ampicillin, ascorbic acid, atorvastatin, azidocillin, aztreonam, bacampicillin, baclofen, benazepril, bendamustine, benzylpenicillin, bezafibrate, biotin, bornaprine, bumetanide, cabastine, canrenoic acid, carbamoylphenoxyacetic acid, carbidopa, carbimazole, carbocisteine, carisoprodol, cefaclor, cefadroxil, cefalexin, cefazoline, cefepim, cefetamet, cefixime, cefotaxime, cefotiam, cefoxitine, cefpodoxime, ceftazidime, ceftibutene, ceftriaxone, cefuroxime, cetirizine, chenodeoxycholic acid, chiorambucil, cidofovir, cilastatine, cilazapril, cinoxacine, ciprofloxacin, cisatracurium besilate, clavulanic acid, clodronic acid, clorazepate, cromoglicic acid, desmeninol, diclofenac, dicloxacillin, enoxacin, eprosartan, ethacrynic acid, etidronic acid, etofylline, etomidate, felbinac, felodipine, fenofibrate, fexofenadine, flavoxate, fleroxacine, flucloxacillin, flufenamic acid, flumazenil, flupirtine, flurbiprofen, fluvastatin, fosfomycin, fosinopril, furosemide, fusidic acid, gabapentin, gemfibrozil, ibandronic acid, ibuprofen, iloprost, imidapril, imipenem, indomethacin, irinotecan, isradipine, ketoprofen, lercanidipine, levodopa, levofloxacin, liothyronine, lipoic acid, lisinopril, lodoxamide, lomefloxacine, lonazolac, loracarbef, loratadine, lovastatin, mefenamic acid, meropenem, mesalazine, metamizole, methotrexate, methyldopa, mezlocillin, moexipril, montelukast, moxifloxacin, mupirocin, naproxen, natamycin, nateglinide, nedocromil, nicotinic acid, nifedipine, nilvadipine, nimodipine, nisoldipine, nitrendipine, norfioxacin, ofloxacin, olsalazine, orotic acid, oxacillin, pamidronic acid, pangamic acid, penicillamine, phenoxymethylpenicillin, pentosan polysulphate, perindopril, pethidine, pipemidic acid, piperacillin, pirenoxine, piretanide, probenecid, proglumid, propicillin, prostaglandins, quinapril, quinaprilate, ramipril, repaglinide, reserpine, risedronic acid, salicylic acid, spirapril, sulbactam, sulfasalazine, sultamicillin, tazarotene, tazobactam, telmisartan, tiagabine, tiaprofenic acid, tilidine, tiludronic acid, trandolapril, tranexamic acid, valproic acid, vigabatrine, vincamine, vinpocetine, zanamivir, zoledronic acid, zopiclone, and their salts, isomers and combinations.

19. The active compound-containing pellet according to claim 1, which is contained in a multiparticulate pharmaceutical form, selected from the group consisting of pellet-containing tablets, minitablets, capsules, sachets, inspissated juices and combinations thereof.

20. A process for the production of an active compound-containing pellet according claim 1 by melt processing, comprising:
mixing the pharmaceutically active substance and the polymer(s) for the polymer matrix, to obtain a mixture; and
maintaining said mixture for at least 10 sec at a temperature of at least 5° C. above the glass transition temperature of the polymer or, in the case of a polymer mixture, based on the polymer having the highest glass transition temperature;
extruding said mixture in an extruder, to obtain an extruded mixture; and
discharging the extruded mixture by die-face cutting with subsequent rounding to give pellets having an average particle size in the range from 300 to 1100 μm; and
coating the pellet by spray application with a polymer coating of an anionic (meth)acrylate copolymer.

21. The process according to claim 20, further comprising:
adding at least one pharmaceutically customary excipient to the polymer matrix and/or the polymer coating in the production of the pellets.

22. The process according to claim 20, wherein a processing temperature in the extruder is 50 to 200° C.

23. A multiparticulate pharmaceutical form, comprising: pellets according to claim 1.

24. A process for the production of a multiparticulate pharmaceutical form which comprises the pellets according claim 1, said process comprising:
mixing the pharmaceutically active substance and the polymer(s) for the polymer matrix, to obtain a mixture; and
maintaining said mixture for at least 10 sec at a temperature of at least 5° C. above the glass transition temperature of the polymer or, in the case of a polymer mixture, based on the polymer having the highest glass transition temperature;
extruding said mixture in an extruder, to obtain an extruded mixture; and
discharging the extruded mixture by die-face cutting with subsequent rounding to give pellets having an average particle size in the range from 300 to 1100 μm; and
coating the pellet by spray application with a polymer coating of an anionic (meth)acrylate copolymer.

25. Active compound-containing pellets, comprising:
a polymer coating of an anionic (meth)acrylate copolymer; and
a pharmaceutically active substance, embedded in a polymer matrix of one or more polymers,
said pellets having
an average particle size in the range from 300 to 1100 μm,
a friability of at most 0.1%, measured using 200 g of pellets in a screening machine having a 200 μm screen, a screening diameter of 20 cm and 1.5 mm shaking amplitude at a shaking frequency of 50 l/sec for 10 min in the presence of six rubber cubes having a 1.8 cm edge length,
with the proviso that the pellets release no more than 10% of the active compound in a release test according to USP in artificial gastric juice at pH 1.2 after 120 min.

26. A method of delayed release of a pharmaceutically active substance, comprising:
administering a pellet according to claim 1 to an organism in need thereof.
